(19) United States
(12) Patent Application Publication
(10) Pub. No.: US 2008/0004693 A1
(43) Pub. Date: Jan. 3, 2008

(54) RESILIENT THIN FILM TREATMENT OF SUPERLASTIC, SHAPE MEMORY, AND HIGHLY FLEXIBLE METAL COMPONENTS

(75) Inventors: Darren R. Burgess, Charlotte, NC (US); Mark S. Wabalas, Huntersville, NC (US); Glenn A. Rupp, Belmont, NC (US); Franz W. Kellar, Gastonia, NC (US); Donald G. Faulkner, Charlotte, NC (US)

Correspondence Address: TREGO, HINES & LADENHEIM, PLLC, 9300 HARRIS CORNERS PARKWAY, SUITE 210, CHARLOTTE, NC, 28269-3797, US

(73) Assignee: BIOMEDFLEX, LLC, Huntersville, NC (US)

(21) Appl. No.: 11/750,954

(22) Filed: May 18, 2007

Related U.S. Application Data
(63) Continuation-in-part of application No. PCT/US06/60308, filed on Oct. 27, 2006.
(60) Provisional application No. 60/596,910, filed on Oct. 28, 2005.

(51) Int. Cl. A61F 2/04

(52) U.S. Cl. 623/1.18

(57) ABSTRACT

A highly flexible component, for example a medical instrument, device, or implant, is treated with a carbon thin film. The thin film acts as a biocompatible corrosion and wear resistant layer over the base material of the component.

### CROSS-REFERENCE TO RELATED APPLICATIONS

This application is a Continuation-In-Part of International Application PCT/US2006/060308 filed Oct. 27, 2006.

### BACKGROUND OF THE INVENTION

This invention relates generally to corrosion and wear resistant thin films and their manufacture, and more particularly to a method for applying these protective thin films to superelastic and shape memory metals and other metals formed into highly flexible geometries.

Various metal alloys are known which exhibit “superelastic” and/or shape memory qualities (geometry restoration). These materials can be elastically deformed to a far greater degree than ordinary alloys. Other materials can be manufactured into special geometries, for example through laser machining, which give them elastic deformability characteristics similar to superelastic materials. Whether through intrinsic properties or special geometries, the high degree of possible elastic deformation attained is especially useful for the construction of medical instruments, devices, and implants.

One category of medical implants is stents, which are small, tubular devices that are implanted into arteries to hold them open so that blood can flow freely through them. A stent is manufactured in an extended condition. It is then collapsed and inserted into the selected artery and moved to the location of a blockage. The stent is then expanded. While stents are effective in opening arteries, they are subject to chemical attack within the body, and the materials from which they are constructed may be incompatible with biological structures.

### BRIEF SUMMARY OF THE INVENTION

These and other shortcomings of the prior art are addressed by the present invention, which according to one aspect provides a component with enhanced biocompatibility and inertness providing protection against corrosion and wear and blood or chemical interaction. The component includes a metallic member having superelastic or shape memory properties, or a combination thereof, and a resilient thin film disposed on the surface of the metallic member, the thin film having: (a) a first layer consisting essentially of carbon in a non-crystalline microstructure; and (b) an adhesion layer adjacent the surface of the metallic member, the adhesion layer comprising carbon and about 2 to about 20 atomic percent of a selected alloying element.

According to another aspect of the invention a medical implant includes: a non-organic functional portion; a lattice structure attached to the functional portion, the lattice structure comprising metallic members having superelastic or shape memory properties, or a combination thereof, and a thin film disposed on an outer surface of the lattice structure. The thin film includes: (a) a first layer consisting essentially of carbon in a non-crystalline microstructure; and (b) an adhesion layer adjacent the outer surface of the lattice structure, the adhesion layer comprising carbon and about 2 to about 20 atomic percent of a selected alloying element. The lattice structure is adapted to serve as a scaffold for the growth and integration of body tissues into the implant.

According to another aspect of the invention, an elastically deformable component with enhanced biocompatibility and inertness providing protection against corrosion and wear and blood or chemical interaction includes: a metallic member; and a resilient thin film disposed on the surface of the metallic member. The thin film includes (a) a first layer consisting essentially of carbon in a non-crystalline microstructure; and (b) an adhesion layer adjacent the surface of the lattice structure, the adhesion layer comprising carbon and about 2 to about 20 atomic percent of a selected alloying element.

According to another aspect of the invention a medical implant includes a non-organic functional portion; a lattice structure attached to the functional portion, the lattice structure comprising metallic members having elastically deformable properties; and a thin film consisting essentially of carbon in a non-crystalline microstructure disposed on an outer surface of the lattice structure. The thin film includes (a) a first layer consisting essentially of carbon in a non-crystalline microstructure; and (b) an adhesion layer adjacent the outer surface of the lattice structure, the adhesion layer comprising carbon and about 2 to about 20 atomic percent of a selected alloying element. The lattice structure is adapted to serve as a scaffold for the growth and integration of body tissues into the implant.

### BRIEF DESCRIPTION OF THE DRAWINGS

The invention may be best understood by reference to the following description taken in conjunction with the accompanying drawing figures in which:

FIG. 1 is a schematic perspective view of a stent treated in accordance with the present invention;

FIG. 2 is a cross-sectional view of a portion of the stent shown in FIG. 1;

FIG. 3 is a schematic side view of a thin film deposition apparatus for use with the present invention;

FIG. 4 is a schematic side view of a synthetic blood vessel treated in accordance with the present invention; and

FIG. 5 is a schematic side view of a replacement heart valve treated in accordance with the present invention.

### DETAILED DESCRIPTION OF THE INVENTION

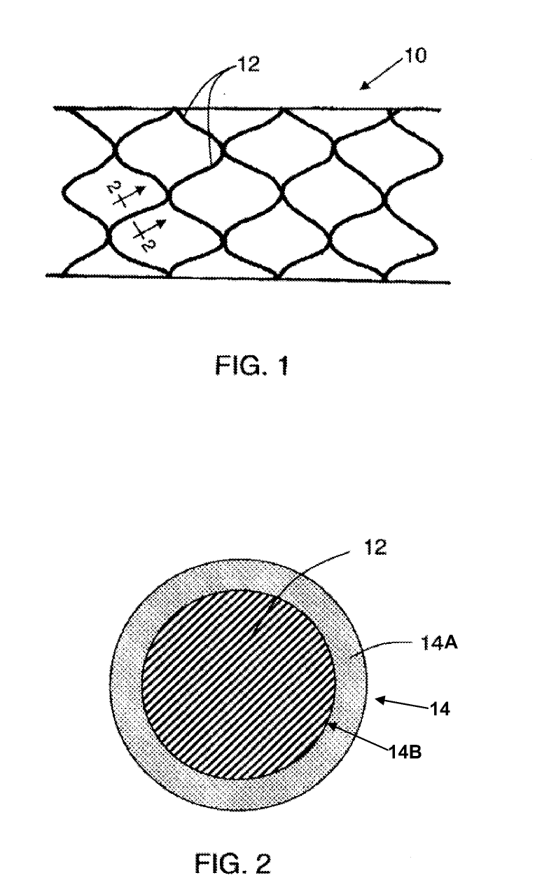

Referring to the drawings wherein identical reference numerals denote the same elements throughout the various views, FIGS. 1 and 2 depict an exemplary stent 10 constructed in accordance with the present invention. The stent 10 has a lattice-like construction of slender, elongated members 12. In the expanded condition, the stent 10 is generally cylindrical. It should be noted that the present invention is equally applicable to other types of implants and components. The stent 10 is made from one or more metals and exhibits elastic deformability characteristics. One example of a suitable material is an alloy of nickel and titanium generally referred to as NITINOL. NITINOL exhibits shape memory characteristics, and when suitably heat treated, also has superelastic properties. Other known metals used for stents, implants, and other medical components include titanium, stainless steels, cobalt chrome, cobalt-chromium-molybdenum, trabecular, titanium-aluminum-niobium and similar materials.

The entire surface of the stent 10 has a thin film 14 of a carbon-based material deposited thereon. This thin film material is essentially pure carbon, has a non-crystalline microstructure, and exhibits a flexural capability with a strain rate of approximately 8% or better. The thin film 14 comprises a carbon structure 14A and an adhesion layer 14B which is disposed adjacent the surface of the stent 10. Together they enable the thin film 14 to endure significant vibration and deformation without cracking or detaching from the substrate or delaminating. Such thin films may be obtained from BioMedFlex LLC, Huntersville, N.C., 28078. A range of parameters and chemicals may be used to form the adhesion layer and carbon-based thin film as described below.

Known materials with elastic deformability characteristics typically exhibit an operational deformation of up to 8%. To the extent that any future materials are developed with elastic and restorative properties in excess of 8%, the thin film 14 will accommodate these materials due to the customizable nature of the thin film 14 and the current exhibited flexural nature and strain rate demonstrated in other applications.

It is believed the thin film 14 is able to reinforce elastically deformable materials. The thin film 14 is sufficiently strong so as to add to the structural integrity and restorative nature of the substrate material. The thin film 14 possesses a high tensile strength so that the thin film 14 forms a composite like structural reinforcement layer on the substrate body.

Similarly, the flexural nature of the thin film 14 produces a skin reinforcement effect that bridges small substrate areas that may constitute weak points or stress risers. The thin film 14 is able to reinforce these locally weak areas and allow the body to better withstand the rigors of low and high cycle fatigue applications by minimizing or reducing the effects of local substrate weak points. The net effect is to hamper the initiation and propagation of surface cracks and defects resulting in an improved or elevated endurance limit and therefore better component integrity

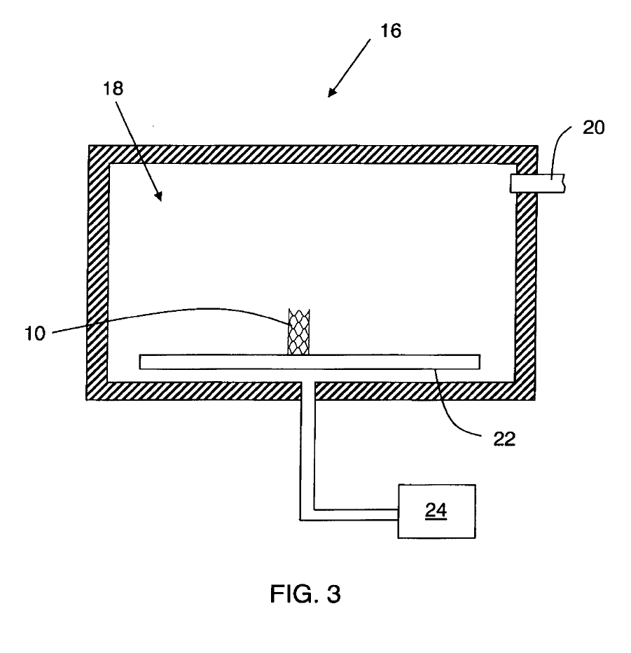

FIG. 3 illustrates a thin film apparatus 16 for applying the thin film 14 to the stent 10. The thin film apparatus 16 is a chemical vapor deposition (CVD) apparatus of a known type. At minimum, it includes a vacuum chamber 18 which receives the workpiece, a hydrocarbon gas source 20, a plasma generator 22 of a known type, and a vacuum pump 24.

The thin film process proceeds as follows. First, the untreated stent 10 is plasma cleaned in a known manner to eliminate any foreign material or contaminants from the surface thereof. The thin film 14 is then deposited all over the exterior of the stent 10 and the members 12 using a plasma assisted chemical vapor deposition (CVD) process. The plasma is specially manipulated so that the thin film material is deposited “around the corner” of the members 12.

An example of a suitable thin film cycle is as follows. The stent 10 is placed in the vacuum chamber 18 which has a base pressure of at most about 1×10⁻⁶ Torr and is capable of operating between 1×10⁻⁵ Torr and 0.1 Torr. The gas of a pure hydrocarbon, such as methane, is flowed into the vacuum chamber 18 at a rate which is determined by a ratio of chamber volume to volumetric flow rate. The ratio should be about 800 minutes or less. During the deposition of the adhesion layer the gas of a second precursor containing at least one of the following elements is flowed into the vacuum chamber: Ti, Al, W, Cr, Si, Ta, Ga, Ge, Hf, Mo, Sr, Zn and Zr. For example, triethylaluminum could be used to introduce Al. The ratio of chamber volume to volumetric flow rate should be about 800 minutes or less. The resulting adhesion layer 14B should contain between about two and about twenty atomic percent of the latter element with the balance being carbon and any other impurity such as hydrogen. To achieve the deposition a plasma is struck in the vacuum chamber 18 as an energy source to drive the deposition processes, such as decomposition of the precursors. The plasma energy should be between about 2 and about 20 watts per square centimeter with respect to the surface area of the stent 10 and associated fixturing. The thickness of the adhesion layer 14B should be at least one complete monolayer and at most the same thickness as the succeeding pure carbon layer.

Following deposition of the adhesion layer 14B, the deposition of the pure carbon layer 14A is achieved by flowing only the pure hydrocarbon into the vacuum chamber 18 and maintaining a plasma as described above. The pure carbon layer thickness can be a factor of about 10 greater than the adhesion layer thickness or more. The pure carbon layer 14A. should be no more than about 5 microns thick.

Once the thin film cycle is complete, the stent 10 is removed from the vacuum chamber 18. Because of the highly flexible nature of the thin film 14, the stent 10 may be inserted into an artery and expanded without cracking or loss of the thin film 14. The finished thin film 14 acts as a biocompatible and inert layer over the base material of the stent 10. The thin film 14 is resistant to scratches and wear and acts as a barrier against biofluids, chemicals, moisture, etc. The thin film 14 exhibits very low surface roughness, which reduces wear and damage to surfaces (e.g. artery walls) in contact with stent 10, causes less buildup and adhesion of other materials, and facilitates extraction because it does not tend to adhere to other materials. The thin film treated stent 10, due to the applied benign surface treatment, acts to impede blood clotting.

It is known to apply anti-inflammatory or antibiotic coatings to the stent 10 to create so-called “drug-eluting” stents. While these coatings are medically effective, they also have a tendency to dissolve, thus exposing the base material of the stent 10. In contrast to the prior art, the stent 10 with the hard carbon thin film 14 will remain protected even when the drug coatings (when and if the two are combined) are no longer present. The resilient, hard carbon thin film also can stand alone as the sole anti-inflammatory surface treatment on a stent.

In addition to stents, it is also possible to apply the thin film to metallic structures fabricated for use as shaped scaffolding and structural support for tissue growth in bioengineered tissue manufacturing and natural tissue regeneration (either externally cultured or internally grown). In the case of structural scaffolding or framework, the thin film would be added to the elastically deformable material to enhance biocompatibility and improve corrosion resistance.

For example, FIG. 4 depicts an exemplary implantable synthetic blood vessel 30. The vessel 30 has a tube portion 32 with a generally cylindrical wall of a biocompatible material, and a scaffold portion 34 attached or incorporated in one end of the tube portion 32. The scaffold portion 34 is configured as a lattice-like construction of slender, elongated members 36. The scaffold portion 34 is made from one or more metals and exhibits elastic deformability characteristics. One example of a suitable material is an alloy of nickel and titanium generally referred to as NITINOL. The entire surface of the scaffold portion 34 has a thin film of a carbon-based material (described above) deposited thereon.

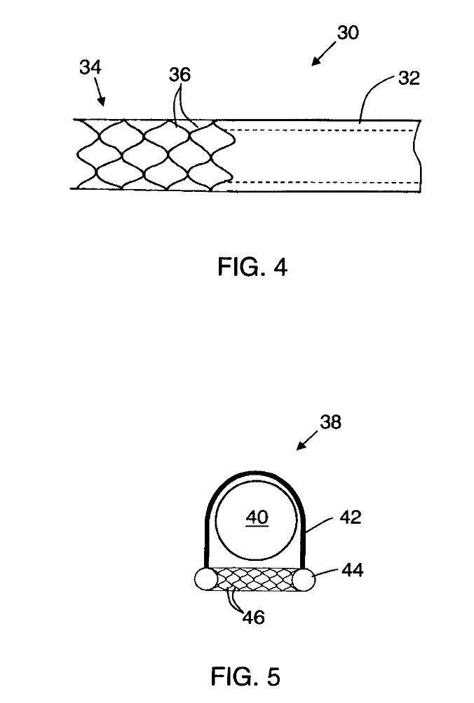

FIG. 5 illustrates an exemplary implantable heart valve 38, which includes a ball 40 captured in a wire-frame cage 42 that is attached to a sealing ring 44. The sealing ring 44 is configured as a lattice-like construction of slender, elongated members 46. The sealing ring 44 is made from one or more metals and exhibits elastic deformability characteristics. One example of a suitable material is an alloy of nickel and titanium generally referred to as NITINOL. The entire surface of the scaffold portion 44 has a thin film of a carbon-based material (described above) deposited thereon.

In use, the scaffold portion 34 of the vessel 30 or the valve 38 would be implanted into a patient. The construction of slender, elongated members forms a support for growth and integration of the body's tissues into the implant. This allows the non-organic functional portion of the implant (i.e. the tube portion 32 or the caged valve ball 40) to be secured fixed in the patient. Antibiotic or anti-inflammatory coatings describe above may also be used in conjunction with the carbon thin film layers.

The foregoing has described a thin-film coated component, apparatus for applying a thin film to such a component, and a method for applying such a thin film. The thin film described herein provides enhanced biocompatibility, improved corrosion protection, reduction of localized release of metal constituents through chemical and mechanical retention, tolerance of normal healing cell growth, and reduced inflammation. While specific embodiments of the present invention have been described, it will be apparent to those skilled in the art that various modifications thereto can be made without departing from the spirit and scope of the invention. Accordingly, the foregoing description of the preferred embodiment of the invention and the best mode for practicing the invention are provided for the purpose of illustration only and not for the purpose of limitation.

What is claimed is:

1. A component with enhanced biocompatibility and inertness providing protection against corrosion and wear and blood or chemical interaction comprising: a metallic member having superelastic or shape memory properties, or a combination thereof; and
a resilient thin film disposed on the surface of the metallic member, the thin film comprising:
(a) a first layer consisting essentially of carbon in a non-crystalline microstructure; and
(b) an adhesion layer adjacent the surface of the metallic member, the adhesion layer comprising carbon and about 2 to about 20 atomic percent of a selected alloying element.

2. The component of claim 1 wherein the thin film has a flexural capability with a strain rate of approximately 8% or better.

3. The component of claim 1 wherein the metallic member comprises an alloy of nickel and titanium.

4. The component of claim 1 wherein the metallic member comprises stainless steel.

5. The component of claim 1 wherein the metallic member comprises cobalt chrome.

6. The component of claim 1 wherein a plurality of members are arranged in a lattice-like structure to form a stent.

7. The component of claim 6 further comprising an antibiotic coating disposed on the thin film.

8. The stent of claim 6 further comprising an anti-inflammatory coating disposed on the thin film.

9. A medical implant, comprising:
a non-organic functional portion;
a lattice structure attached to the functional portion, the lattice structure comprising metallic members having superelastic or shape memory properties, or a combination thereof, and
a thin film disposed on an outer surface of the lattice structure, the thin film comprising:
(a) a first layer consisting essentially of carbon in a non-crystalline microstructure; and
(b) an adhesion layer adjacent the outer surface of the lattice structure, the adhesion layer comprising carbon and about 2 to about 20 atomic percent of a selected alloying element;
wherein the lattice structure is adapted to serve as a scaffold for the growth and integration of body tissues into the implant.

10. The implant of claim 9 wherein the thin film has a flexural capability with a strain rate of approximately 8% or better.

11. The implant of claim 9 wherein the lattice structure comprises an alloy of nickel and titanium.

12. The component of claim 9 wherein the lattice structure comprises stainless steel.

13. The component of claim 9 wherein the lattice structure comprises cobalt chrome.

14. The implant of claim 9 further comprising an antibiotic coating disposed on the thin film.

15. The implant of claim 9 further comprising an anti-inflammatory coating disposed on the thin film.

16. The implant of claim 9 wherein the functional portion is a caged ball heart valve structure.

17. The implant of claim 9 wherein the functional portion is a synthetic blood vessel.

18. An elastically deformable component with enhanced biocompatibility and inertness providing protection against corrosion and wear and blood or chemical interaction comprising:
a metallic member; and
a resilient thin film disposed on the surface of the metallic member, the thin film comprising:
(a) a first layer consisting essentially of carbon in a non-crystalline microstructure; and
(b) an adhesion layer adjacent the surface of the lattice structure, the adhesion layer comprising carbon and about 2 to about 20 atomic percent of a selected alloying element.

19. The component of claim 18 wherein the thin film has a flexural capability with a strain rate of approximately 8% or better.

20. The component of claim 18 wherein the metallic member comprises an alloy of nickel or chrome or titanium or ferrous metal (Glenn check here for material constituents).

21. The component of claim 18 wherein a plurality of members are arranged in a lattice-like structure.

22. The component of claim 21 wherein the lattice-like structure defines a stent.

23. The component of claim 18 wherein the thin film has a flexural capability with a strain rate of approximately 8% or better.

24. The component of claim 18 wherein the lattice structure comprises an alloy of nickel or titanium or a combination thereof

25. The component of claim 18 wherein the lattice structure comprises stainless steel.

26. The component of claim 18 wherein the lattice structure comprises cobalt chrome.

27. The component of claim 18 further comprising an antibiotic coating disposed on the thin film.

28. The component of claim 18 further comprising an anti-inflammatory coating disposed on the thin film.

29. A medical implant, comprising:
a non-organic functional portion;
a lattice structure attached to the functional portion, the lattice structure comprising metallic members having elastically deformable properties; and
a thin film consisting essentially of carbon in a non-crystalline microstructure disposed on an outer surface of the lattice structure, the thin film comprising:
(a) a first layer consisting essentially of carbon in a non-crystalline microstructure; and
(b) an adhesion layer adjacent the outer surface of the lattice structure, the adhesion layer comprising carbon and about 2 to about 20 atomic percent of a selected alloying element;
the lattice structure adapted to serve as a scaffold for the growth and integration of body tissues into the implant.

30. The implant of claim 29 wherein the thin film has a flexural capability with a strain rate of approximately 8% or better.

31. The implant of claim 29 wherein the lattice structure comprises an alloy of nickel or titanium or a combination thereof.

32. The component of claim 29 wherein the lattice structure comprises stainless steel.

33. The component of claim 29 wherein the lattice structure comprises cobalt chrome.

34. The implant of claim 29 further comprising an antibiotic coating disposed on the thin film.

35. The implant of claim 29 further comprising an anti-inflammatory coating disposed on the thin film.

36. The implant of claim 29 wherein the functional portion is a caged ball heart valve structure.

37. The implant of claim 29 wherein the functional portion is a synthetic blood vessel.
